(19) United States
(12) Patent Application Publication
(10) Pub. No.: US 2008/0108972 A1
(43) Pub. Date: May 8, 2008

(54) UNIVERSAL CUTTER FOR GUIDE CATHETERS

(75) Inventors: Christopher C. Andrews, Lake Elsinore, CA (US); Kirk Svalstad, Lake Elsinore, CA (US); Brad Kellerman, Escondido, CA (US); Devon Brown, San Diego, CA (US)

Correspondence Address: FAEGRE & BENSON, LLP;BOSTON SCIENTIFIC PATENT DOCK, 2200 WELLS FARGO CENTER, 90 SOUTH SEVENTH STREET, MINNEAPOLIS, MN, 55402-3901, US

(73) Assignee: Cardiac Pacemakers, Inc., St. Paul, MN (US)

(21) Appl. No.: 11/854,768

(22) Filed: Sep. 13, 2007

Related U.S. Application Data
(60) Provisional application No. 60/864,901, filed on Nov. 8, 2006.

(51) Int. Cl. A61M 25/00

(52) U.S. Cl. 604/523

(57) ABSTRACT

A universal cutter for catheters includes a lead fixation feature having a deflectable lower portion and a hinge portion. The deflectable lower portion and the hinge portion of the lead fixation feature allow the cutter to hold and secure leads having varying outer diameters. The cutter holds the lead in place while a guide catheter is moved in a proximal direction toward a blade on the cutter to be split from about the lead.

### CROSS REFERENCE TO RELATED APPLICATIONS

The present application claims the benefit of U.S. Provisional Application No. 60/864,901 filed Nov. 8, 2006, entitled “Universal Cutter for Guide Catheters,” which is incorporated herein by reference.

### TECHNICAL FIELD

The present invention relates generally to devices for removing guide catheters from leads. More specifically, the present invention relates to universal, notched cutters for slicing and removing guide catheters from implanted leads having multiple diameters.

### BACKGROUND

Implantable medical devices, such as pacemakers or other cardiac rhythm management devices, often require that a lead be implanted within the body of a patient to connect the device with a specific portion of a patient's body, such as the heart. Minimally invasive techniques have been developed to permit such leads to be implanted within the patient's body. One example of such a minimally invasive technique is to utilize a guide catheter, requiring only a relatively small incision at the insertion site. Once the implanted lead has been positioned in the patient's body, the catheter must be removed without displacing the lead from its implantation position. Typically, the catheter must be cut or split as it is retracted from the patient's body. Thus, improvements to the tools and techniques used to remove a guide catheter from a patient's body after lead implantation are desired.

### SUMMARY

The present invention, in one embodiment, is a device for removing a guide catheter from about an elongate medical electrical lead positioned within a lumen of the guide catheter. The device comprises a body, a lead engagement feature, a blade, and a lead shield. The body is configured to be gripped by a clinician and includes a generally linear lower edge, a proximal end portion and a distal end portion. The lead engagement feature has a generally elliptical transverse cross-sectional shape and is configured to frictionally engage the elongate medical electrical lead and to inhibit longitudinal movement of the lead relative to the device. The blade extends from the distal end portion of the body and includes a distal cutting edge and a lower edge. The lead shield is attached to the lower edge of the blade and has a lower surface configured to contact the outer surface of the lead.

In another embodiment, the present invention is a device for removing a guide catheter from about an elongate medical electrical lead positioned within a lumen of the guide catheter, lead having an outer diameter ranging from about 3 French to about 8 French. The device comprises a body, means attached to the body for receiving and frictionally engaging the lead and inhibiting longitudinal movement of the lead relative to the body, a blade, and a lead shield. The blade has a distal cutting edge and a lower edge. The lead shield is attached to the lower edge of the blade and includes a lower surface configured to contact the outer surface of the lead.

In yet another embodiment, the present invention is a system comprising a guide catheter, a medical electrical lead, and a cutter. The guide catheter is configured to access a coronary sinus of a patient and includes a lumen therethrough. The medical electrical lead has an outer diameter of from about 3 French to about 8 French and is disposed in the lumen of the guide catheter. The cutter is configured to remove the guide catheter from about the medical electrical lead. The cutter includes a body, a lead engagement, a blade, and a lead shield. The lead engagement feature is attached to the body and includes an upper portion, a lower portion, and a hinge therebetween. The hinge is configured to permit deflection of the lower portion to permit the lead to be received between the upper and lower portions and thereafter to cause the lower portion to impart a radial force on the lead such that the upper and lower portions frictionally engage the lead and inhibit longitudinal movement of the lead relative to the body. The blade has a distal cutting edge and a lower edge, the distal cutting edge configured to cut the catheter as it is moved proximally relative to the body and the lead. The lead shield is attached to the lower edge of the blade and includes a lower surface configured to contact the outer surface of the lead. The lead shield is configured such that it can be inserted between the lead and the catheter.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is an elevation view of a universal guide catheter cutter in use for removing a guide catheter from about a medical electrical lead according to one embodiment of the present invention.

FIG. 2 is a detailed elevation view of the cutter of FIG. 1 engaging a medical electrical lead according to one embodiment of the present invention.

FIGS. 3A and 3B are perspective and elevation views, respectively, of the cutter of FIG. 1.

FIG. 4A is a partial cross-sectional view of the cutter of FIG. 1 taken along the line 4A-4A in FIG. 3B.

FIG. 4B is a cross-sectional detail of the lead engagement feature of the cutter of FIG. 1.

FIG. 4C is a partial cross-sectional view of the cutter of FIG. 1 taken along the line 4C-4C in FIG. 3B.

FIG. 5 is a detailed elevation view of the blade portion of the cutter of FIG. 1.

FIG. 6A is a perspective view of the cutter of FIG. 1 shown in use according to an embodiment of the present invention.

FIG. 6B is an enlarged perspective view of the cutter as illustrated in FIG. 6A.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

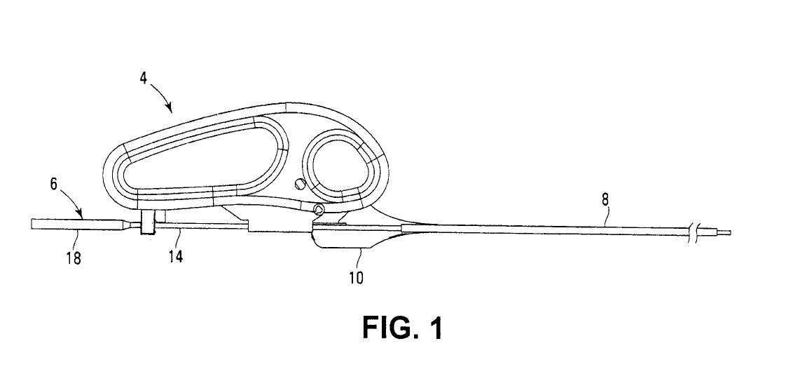

FIG. 1 shows a cutter 4 engaging a lead 6 inserted within a central axial lumen of a guide catheter 8 according to an embodiment of the present invention. As shown in FIG. 1, a proximal end of a guide catheter 8 includes a fitting 10. The fitting 10 can be any appropriate proximal fitting as is known in the art. For example, the proximal fitting 10 can include a luer hub so as to allow a physician to manipulate the guide catheter 8 and/or connect a device such as a hemostasis valve to the guide catheter 8. In one embodiment of the invention, as shown in FIG. 1, the fitting 10 can be a conventional cuttable luer hub.

In order to keep the diameter of the guide catheter 8 as small as possible to minimize the invasiveness of the placement procedure, and to reduce blood back pressure during placement, the central axial lumen of the catheter 8 is sized to closely fit about the lead 6. Typically, a cardiac lead body 14 is smaller in diameter than a proximal end 18 of the lead 6. As such, the catheter 8 needs to be split lengthwise in order to remove the catheter 8 from about the lead 6. The cutter 4 operates to split the catheter lengthwise to permit the removal of the guide catheter 8 from about the lead 6 without disturbing or displacing the position of the lead 6 from its location within a patient's body.

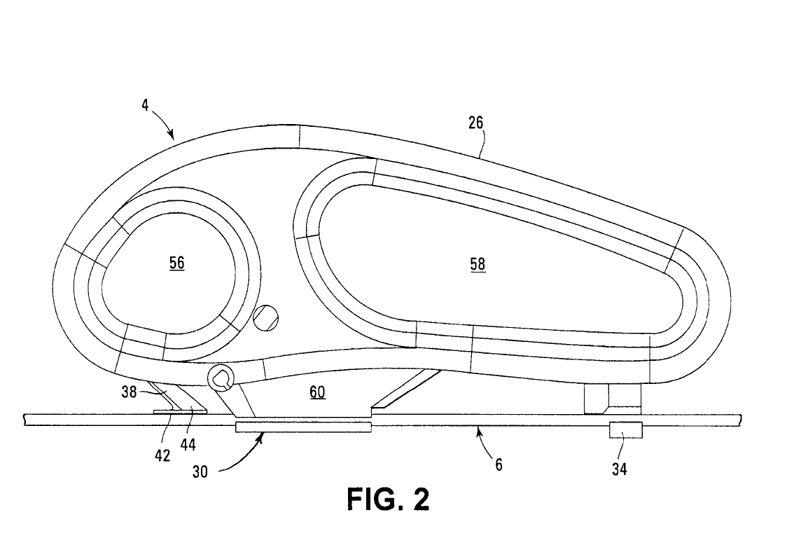

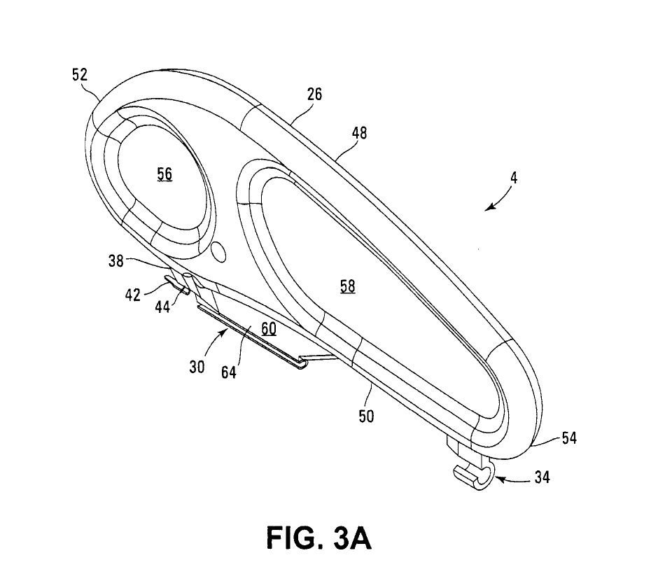

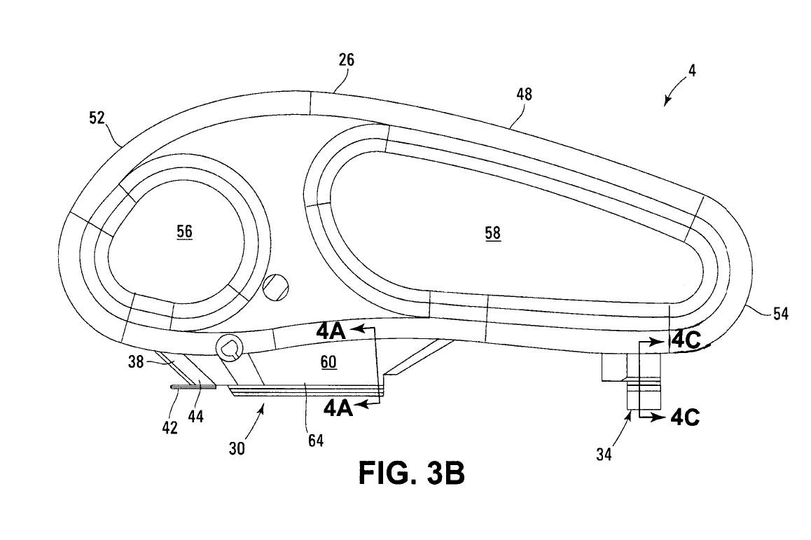

FIG. 2 is a detailed elevation view of the cutter 4, and FIGS. 3A and 3B are perspective and elevation views, respectively, of the cutter 4. According to the illustrated embodiment, the universal guide catheter cutter 4 includes a body 26, a lead engagement feature 30, a lead management feature 34, a blade 38, and a lead shield 42 attached to a lower edge 44 of the blade 38. As shown in FIGS. 3A and 3B, the body 26 includes an upper edge 48, a lower edge 50, a front edge 52, and a rear edge 54. The body 26 can be made from a wide variety of materials including, but not limited to, plastics, polymers, elastomers, resins, and combinations thereof. According to one embodiment of the present invention, the body 26 is injection molded. In another embodiment of the present invention, the body 26 is made of polyether block amide sold under the brand name Pebax®. In one embodiment, the body 26 is made of Pebax® 63D, which has a Shore durometer hardness of about 63. Located on either side of the body 26 are a pair of gripping recesses 56 and 58, which assist the clinician in securely holding the cutter 4. Extending from the lower edge 50 is a web 60 having a lower portion 64. As shown, the web 60 and the body 26 operate in part to support the blade 38. The lead engagement feature 30 is located opposite the body 26 adjacent to the web 60. The lead management feature 34 extends from the lower edge 50 and is adjacent to the rear edge 54.

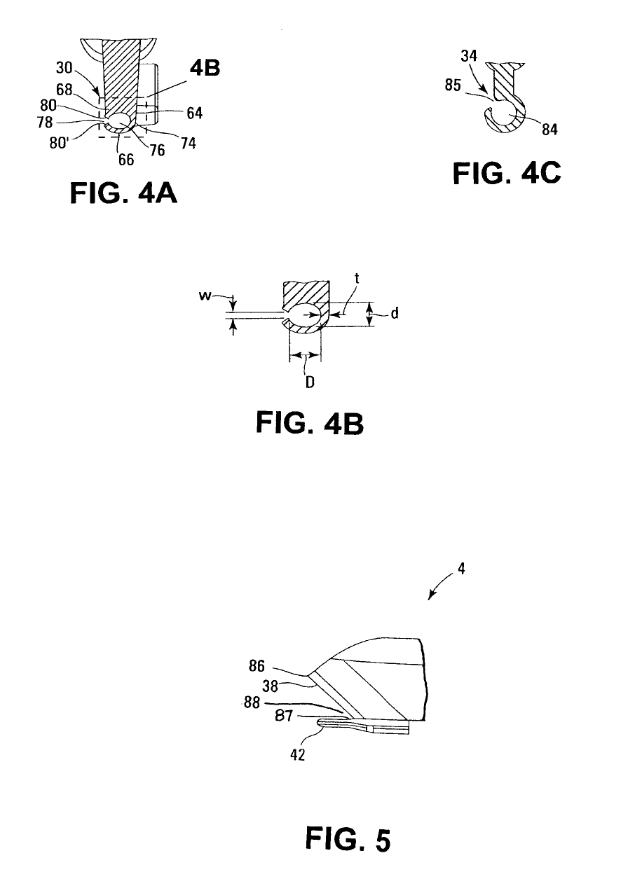

FIG. 4A is a partial cross-sectional view of the cutter 4 taken along the line 4A-4A in FIG. 3B. According to the illustrated embodiment, the lead engagement feature 30 has a generally elliptical cross-sectional shape and includes a lower portion 66, an upper portion 68 adjacent to and extending from the lower portion 64 of the web 60, and a hinge portion 74 between the lower and upper portions 66, 68. As further shown in FIG. 4A, the lower, upper, and hinge portions 66, 68, and 74 define an inner opening 76 and an open side 78 opposite the hinge portion 74. The open side 78 can open to the right side or to the left side of the cutter 4.

The lower portion 66 is deflectable at the hinge portion 74 to permit a portion of a lead to be received within the lead engagement feature 30 between the lower and upper portions 66, 68. The lead engagement feature 30 also includes opposed retaining lips 80, 80′ extending radially inward from the upper and lower portions 68, 66, respectively, adjacent the open side 78. The retaining lips 80, 80′ are configured to substantially inhibit the lead 6 from being ejected from the open side 78.

In operation, the lead engagement feature 30 operates to frictionally engage and hold the lead 6 in place relative to the cutter 4 as the guide catheter 8 is removed from about the lead 6 (see FIGS. 1 and 2). The hinge portion 74 is configured to permit deflection of the lower portion 66 away from the upper portion 68, such that the lead 6 can be inserted through the open side 78 and received between the lower and upper portions 66, 68. The hinge portion 74 is further configured to cause the lower portion 66 to impart sufficient radial force on the lead 6 so as to frictionally engage the lead 6 between the upper and lower portions 68, 66 and substantially prevent longitudinal movement of the lead 6 relative to the cutter 4.

The novel elliptical cross-sectional configuration of the lead engagement feature 30 further provides for a cutter 4 that can be used in conjunction with a range of lead sizes. Moreover, the elliptical lead engagement feature 30 with the hinge portion 74 can effectively engage multiple lead sizes without requiring the application of an additional external clamping force, such as pressure applied by the clinician using his or her fingers. In one embodiment, the lead engagement feature 30 is configured to receive and retain leads having outer diameters ranging from about 3 French to about 8 French. In another embodiment, the lead engagement feature 30 is configured to receive and retain leads having outer diameters ranging from about 4 French to about 6 French. Thus, a separate cutter 4 for each size of lead need not be provided during a given procedure.

FIG. 4B is a cross-sectional detail of the lead engagement feature 30. As shown in FIG. 4B, the elliptical cross-sectional shape of the lead engagement feature 30 defines a major dimension D and a minor dimension d, and the side opening 85 has a width W when in an undeflected state. In various exemplary embodiments, major dimension D of the elliptical cross-sectional shape of the lead engagement feature 30 ranges from about 0.062 inches to about 0.078 inches, the minor dimension d ranges from about 0.046 inches to about 0.054, and the width W of the open side 78 ranges from about 0.012 inches to about 0.018 inches when undeflected. In one embodiment, the lead engagement feature 30 has a major dimension D of about 0.070 inches and a minor dimension d of about 0.050 inches, and the open side 78 has a width W of about 0.015 inches when undeflected. Of course, in other embodiments, these features may have other dimensions, depending on the desired functionality and the range of lead sizes to be accommodated. In one embodiment, the lead engagement feature 30 is made from Pebax® 63D, and the hinge portion 74 has a thickness t of about 0.020 inches near its apex, although in other embodiments, other configurations and materials may be utilized for the lead engagement feature 30 to provide the desired functionality.

FIG. 4C is a cross-sectional view of the cutter 4 taken along the line 4C-4C in FIG. 3B showing the proximal lead management feature 34. The proximal lead management feature 34, according to an embodiment of the present invention, defines a central opening 84. The central opening 84 is generally axially aligned with the inner opening 76 of the lead engagement feature 30. A side opening 85 allows the lead 6 to be snapped into the opening 84 from the side. The side opening 85 can open to either side of the cutter 4. In one embodiment, the side opening 85 of the lead management feature 34 opens to the same side as the open side 78 on the lead engagement feature 30 (see FIGS. 3A and 3B). According to a further embodiment of the present invention, the lead management feature 34 is made of a resilient deformable material which permits entry of the lead body 14 through the side opening 85. The lead management feature 34 assists the cutting procedure by managing or holding any extra lead length out of the way during cutting.

FIG. 5 is a partial side view of the cutter 4 showing a detailed view of the blade 38 according to an embodiment of the present invention. As shown in FIG. 5, the blade 38 includes an upper cutting edge 86 and a lower cutting edge 87. A notch 88 is defined between the upper cutting edge 86 and lower cutting edge 87. As shown, the upper and lower cutting edges 86, 87 extend distally from the notch 88. The notch 88 thus defines the rearmost cutting portion of blade 38. According to one embodiment, the notch 88 is positioned adjacent to the lead shield 42. The positioning of the notch 88 as the rearmost cutting portion of the blade 38 provides improved resistance to binding during cutting or splitting of a catheter from about a lead.

The lower cutting edge 87 of the blade 38 adjacent to the lead shield 42 is angled forward from the notch 88 to aid in insertion of the blade 38 within the lumen of a catheter. Above the notch 88, the upper cutting edge 86 is angled to prevent the catheter from climbing up the blade 38 away from the lead shield 42, which can cause the cutter 4 to bind. According to one embodiment of the invention, the blade 38 can be positioned within the cutter 4 by having the body 26, the web 60, and the lead shield 42 molded about the blade 38 (see FIG. 2), provided the cutter 4 is made of a moldable material. Other methods of construction and assembly may also be used for the cutter 4. The configuration of the notch 88 also improves the ability of the cutter 4 to split catheters having a reinforcing substrate such as a mesh or braid embedded within the catheter body. Additionally, the configuration of the notch 88 also allows the cutter 4 to restart the cutting of a catheter body after stopping at an intermediate point between a proximal end and a distal end of the catheter.

As further shown in FIGS. 3A, 3B, and 5, the lead shield 42 is attached to the lower edge 44 of the blade 38 and generally extends from the front of the web 60 forward of the blade 38. In one embodiment, the lead shield 42 is provided as a separate element from the blade 38 and may be secured to the blade 38 by any attachment technique known in the art (e.g. welding, soldering, brazing, or adhesive). According to one embodiment of the present invention, the lead shield 42 is laser-welded to the blade 38. The lead shield 42 can be made from a variety of suitable materials that are capable of forming a cutting edge including, but not limited to, the following: any grade of stainless steel, titanium, titanium alloys, ceramics, metallized plastics, powdered metals, and combinations thereof. In one embodiment of the invention, the lead shield 42 is a stainless steel lead shield 42. In one embodiment, the lower edge of the lead shield 42 has a concave shape that is complimentary to the generally circular cross-sectional shape of the lead body 14.

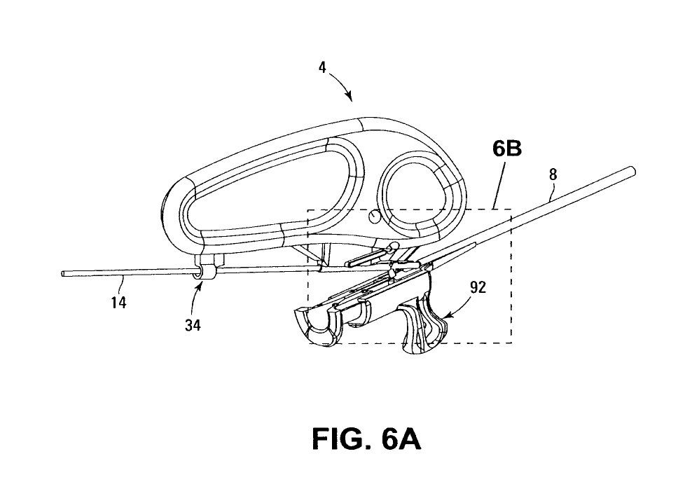

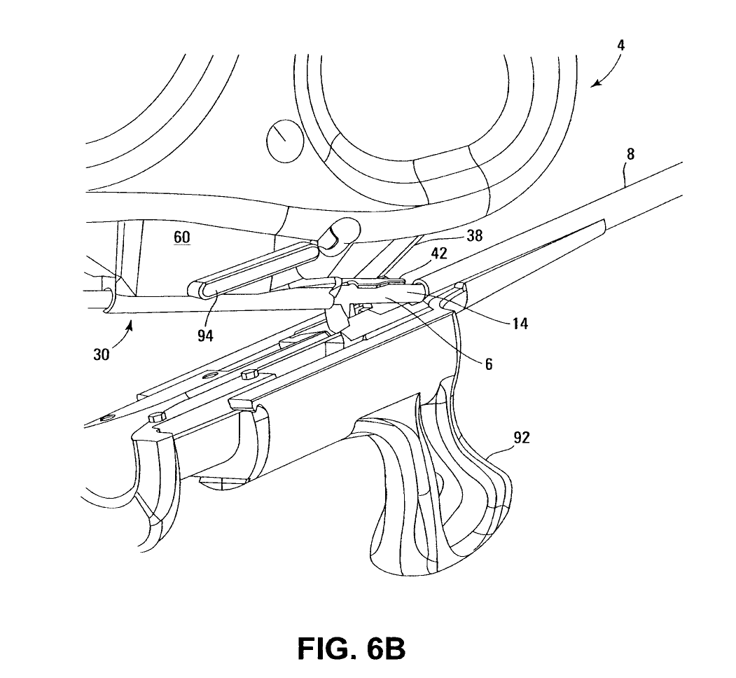

FIGS. 6A and 6B are perspective views of the catheter 4 shown in conjunction with a splittable hub 92 such as is described in co-pending U.S. Provisional Patent application No. 60/864,895 entitled Break-Away Hemostasis Hub, the contents of which are incorporated herein by reference. According to the embodiment shown in FIGS. 6A and 6B, the web 60 also includes at least one optional wing 94 which can be located on either side of the web 60. The optional wing 94 keeps the hub 92 from interfering with the cutting of the catheter 8 by forcing the hub 92 out of the cutting plane during the cutting procedure. The universal cutter 4 frictionally engages and holds the lead 6 in place while the guide catheter 8 is moved in a proximal direction towards the blade 38. The lead shield 42, attached to the lower edge 44 of the blade 38, generally complements the outer surface of the lead body 14 and contacts the lead body 14 between the outer surface of the lead body 14 and the inner surface of the guide catheter 8 to be split. The lead shield 42 assists the orientation of the catheter 8 with respect to the blade 38 as the catheter 8 is moved in a proximal direction towards the blade 38 during the splitting process. As the guide catheter 8 engages the blade 38, it begins to split. The lead shield 42 helps deflect the split portions of the guide catheter 8 away from the cutter 4.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A device for removing a guide catheter from about an elongate medical electrical lead positioned within a lumen of the guide catheter, the device comprising:
a body configured to be gripped by a clinician and including a generally linear lower edge, a proximal end portion and a distal end portion;
a lead engagement feature having a generally elliptical transverse cross-sectional shape and being configured to frictionally engage the elongate medical electrical lead and to inhibit longitudinal movement of the lead relative to the device;
a blade extending from the distal end portion of the body and including a distal cutting edge and a lower edge; and
a lead shield attached to a lower edge of the blade, the lead shield having a lower surface configured to contact the outer surface of the lead.

2. The device of claim 1 wherein the lead engagement feature includes an upper portion attached to the lower edge of the body, a lower portion, and a hinge portion between the upper and lower portions.

3. The device of claim 2 further comprising an elongate web extending between the lower edge of the body and the upper portion of the lead engagement feature.

4. The device of claim 3 and further comprising a proximal lead management feature attached to the web proximal to the lead engagement feature and including an side opening sized to receive a portion of the lead.

5. The device of claim 1 wherein the lead engagement feature is configured to engage a lead having an outer diameter ranging in size from about 3 French to about 8 French.

6. The device of claim 5 wherein the lead engagement feature is configured to engage a lead having an outer diameter ranging in size from about 4 French to about 6 French.

7. The device of to claim 1 wherein the lead management feature and the lead engagement feature are substantially coaxially aligned.

8. The device of claim 1 wherein the body is made from a material including at least one of a plastic, a polymer, an elastomer, and a resin.

9. The device of claim 8 wherein the body is made substantially of polyether block amide having a hardness of about 63D on the Shore hardness scale.

10. The device of claim 1 wherein the lower portion of the lead engagement feature is deflectable at the hinge portion to permit a portion of the lead to be received within the lead engagement feature between the upper and lower portions.

11. The device of claim 10 wherein the lead engagement feature further includes:
an open side opposite the hinge portion; and
opposed retaining lips extending radially inward from the upper and lower portions adjacent the open side, the retaining lips configured to substantially inhibit the lead from being ejected from the open side when received between the upper and lower portions of the lead engagement feature.

12. The device of claim 1 wherein the lead shield generally extends forward of the blade.

13. The device of claim 12 wherein the lead shield is provided as a separate element from the blade and is attached to the blade by a weld joint.

14. The device of claim 12 wherein the lead shield is made substantially from at least one of stainless steel, titanium, titanium alloy, ceramic, metallized plastic, and powdered metal.

15. The device of claim 1 wherein the distal cutting edge includes an upper cutting edge, a lower cutting edge, and a notch therebetween, and wherein the lower cutting edge is positioned adjacent the lead shield and angled forward from the notch.

16. A device for removing a guide catheter from about an elongate medical electrical lead positioned within a lumen of the guide catheter, lead having an outer diameter ranging from about 3 French to about 8 French, the device comprising:
a body;
means attached to the body for receiving and frictionally engaging the lead and inhibiting longitudinal movement of the lead relative to the body;
a blade extending from the body and having a distal cutting edge and a lower edge; and
a lead shield attached to the lower edge of the blade and including a lower surface configured to contact the outer surface of the lead.

17. The device of claim 16, wherein distal cutting edge of the blade includes a lower cutting edge and an upper cutting edge with a notch therebetween, wherein the lower cutting edge is adjacent the lead shield and angled forward from the notch.

18. The device of claim 16 wherein the lead shield is configured such that it can be inserted between the lead and the catheter when the lead is engaged by the lead engagement feature, and wherein the blade is configured to cut the catheter as the catheter is moved proximally relative to the blade and the lead.

19. A system comprising:
a guide catheter configured to access a coronary sinus of a patient and including a lumen therethrough;
a medical electrical lead having an outer diameter of from about 3 French to about 8 French disposed in the lumen of the guide catheter; and
a cutter configured to remove the guide catheter from about the medical electrical lead, the cutter including:
a body;
a lead engagement feature attached to the body and including an upper portion, a lower portion, and a hinge therebetween, the hinge configured to permit deflection of the lower portion to permit the lead to be received between the upper and lower portions and thereafter to cause the lower portion to impart a radial force on the lead such that the upper and lower portions frictionally engage the lead and inhibit longitudinal movement of the lead relative to the body;
a blade extending from the body and having a distal cutting edge and a lower edge, the distal cutting edge configured to cut the catheter as it is moved proximally relative to the body and the lead; and
a lead shield attached to the lower edge of the blade and including a lower surface configured to contact the outer surface of the lead, the lead shield configured such that it can be inserted between the lead and the catheter.

20. The device of claim 19 wherein the lead engagement feature has a generally elliptical cross-sectional shape.
